(19) United States
(12) Patent Application Publication
(10) Pub. No.: US 2013/0230674 A1
(43) Pub. Date: Sep. 5, 2013

(54) MASK STRAP HEAD, FACIAL OR EAR CUSHION

(71) Applicants: James N. Curti, Bakersfield, CA (US); Walter R. Van Horn, Lancaster, CA (US); Stephen Mark Higgins, Carrollton, TX (US)

(72) Inventors: James N. Curti, Bakersfield, CA (US); Walter R. Van Horn, Lancaster, CA (US); Stephen Mark Higgins, Carrollton, TX (US)

(73) Assignee: SALTER LABS, ARVIN, CA (US)

(21) Appl. No.: 13/823,940

(22) PCT Filed: Sep. 16, 2011
(86) PCT No.: PCT/US2011/051949

§ 371 Date: May 14, 2013

Related U.S. Application Data
(60) Provisional application No. 61/383,980, filed on Sep. 17, 2010.
(60) Provisional application No. 61/405,422, filed on Oct. 21, 2010.

(51) Int. Cl. A61M 16/06

(52) U.S. Cl. CPC A61M 16/06
USPC 428/34.1; 428/343

(57) ABSTRACT

An head, facial or ear cushion for mounting to a strap of a respiratory or traditional device. The head, facial or ear cushion comprises an elongate flattened body which defines an internal cavity for accommodating a strap of a respiratory or traditional device and generally has a flattened oval shape forming two elongate opposed exterior surfaces. The two opposed surfaces are generally planar and extend substantially parallel to one another so that a width of the head, facial or ear cushion is at least twice a thickness of the head, facial or ear cushion. The body is configured so as to restrict axial movement of the body along the strap, during use. In one embodiment, the body is folded into a folded configuration, about a longitudinal centerline, to define the internal cavity which at least partially encloses while in another embodiment, the body is generally oval in shape and has an exterior surface which extends completely around and encases the strap.

### FIELD OF THE INVENTION

The present invention relates to head, facial or ear cushions for medical respiratory or traditional devices and, more specifically, to head, facial or ear cushions for preventing irritation to a patient's ears caused by straps securing the respiratory or traditional device to the face of the patient adjacent the region of the patient's mouth and/or nose.

### BACKGROUND OF THE INVENTION

A number of medical respiratory procedures require the prolonged positioning of a respiratory or other device, such as a mask or cannula, in the region adjacent to or covering either a patient's nose and/or mouth. Typical applications for such devices include, for example, detecting a patient's respiratory cycles and exhaled CO₂ levels, such as in detecting and monitoring sleep apnea, or the provision of supplemental or therapeutic gas, such as air or oxygen, to a patient during treatment of a patient with sleep apnea.

The respiratory or traditional devices commonly used in such medical procedures, e.g., for treating a patient with sleep apnea, are typically secured onto a patient by at least one strap(s), which is typically an elastic strap, and that strap extends from the respiratory or traditional device and around the patient's head in the region of the top portion of the patient's ears, with the strap generally passing just over the upper curvature of the patient's ears, generally between an ear flap of the outer ear or auricle and the side of the patient's head. When treating a patient with sleep apnea, this typically requires that the patient wear the respiratory mask or some other device continuously for an extended period of time—typically about 6-11 hours each night so that the patient may sleep. Such extended and repeated wearing of a respiratory mask, or some other device, often results in discomfort to the patient and irritation to the patient's ears and skin due to rubbing of the strap(s), particularly in the region where the strap passes over and contacts the upper curvature of the patient's ears generally between the ear flap and the side of the patient's head.

While prior pads have been employed in the art, such pads are generally round or circular and, in order to provide the desired cushioning, tend to be fairly bulky and, in turn, leads to discomfort to the patient. Furthermore, none of the known pads, due to their bulkiness, can be comfortably accommodated about the curvature of the ear, between the ear flap and the side of the patient's head.

The present invention provides a solution to these and related problems of the prior art.

### SUMMARY OF THE INVENTION

The present invention is directed to a head, facial or ear cushion for mounting to a strap of a respiratory or traditional device, either before of after the strap is attached to the mask, and generally includes an elongate, generally flattened, oval body which provides a cushioning effect for the patient in the region where the strap passes over and contacts the upper curvature of the patient's outer ears to improve comfort to the patient and reduce irritation to the patient's ears and skin due to pressure and/or rubbing of the strap(s).

According to the present invention, the head, facial or ear cushion may be positioned either adjacent an upper portion of the outer ear, adjacent a cheek or behind the back of the head in order to provide comfort to a patient utilizing a mask, or some other breathing apparatus, in a comfortable and streamlined manner.

Another object of the invention is to provide a head, facial or ear cushion which is generally much thinner than it is length and its width, e.g., the head, facial or ear cushion generally has a “flattened” oval shape which results in two elongate opposed exterior surfaces of the head, facial or ear cushion that are generally planar and extend parallel to one another so that the width of the head, facial or ear cushion is at least twice, and preferably about three to four times greater than the thickness of the head, facial or ear cushion. Due to such flattened arrangement, the two elongate opposed exterior surfaces can be readily accommodated, during use, between the upper portion of the outer ear lobe of the patient and the side of the patient's head, adjacent a cheek, or behind the back of the head without causing discomfort.

Still another object of the invention is to provide the head, facial or ear cushion with a thickness which is designed so that the head, facial or ear cushion can be lightly, but comfortably, sandwiched between the upper portion of the lobe of the outer ear of the patient and the side of the patient's head while still assisting with avoiding axial movement of the head, facial or ear cushion along the strap, during use.

Yet another object of the invention is to secure the head, facial or ear cushion in a region of the strap bearing against an upper curvature of the outer ear, generally between an ear flap of the outer ear and the side of the patient's head, by an adhesive strip, which generally includes a removable layer, covering the adhesive strip, which is to be removed prior to securing the head, facial or ear cushion about the strap.

The invention also relates to an head, facial or ear cushion for mounting to a strap of a respiratory or traditional device, the head, facial or ear cushion comprising an elongate flattened body defining an internal cavity for accommodating a strap of a respiratory or traditional device, the elongate body being configured so as to restrict axial movement of the elongate body along the strap, during use, and maintain the head, facial or ear cushion substantially centered with respect to an upper curvature of an outer ear, generally between the ear flap and the side of the patient's head.

According to one embodiment, the body is initially folded into a folded configuration, about a longitudinal centerline of the body, to define the internal cavity and at least partially enclose the strap of the respiratory or traditional device. The head, facial or ear cushion is then retained in its folded configuration by an adhesive engaging with opposed inwardly facing surfaces of the folded body adjacent the strap.

According to another embodiment, the flattened body is generally a flattened oval shape which has a continuous exterior surface which extends continuously and completely around the strap so as to encase the strap, and the internal cavity extends completely through the body and is defined by the exterior surface for accommodating the strap.

In a further embodiment, the continuous exterior surface of the body of the head, facial or ear cushion forms opposing facing first and second exterior sidewall surfaces and at least one of the exterior sidewall surfaces includes a plurality of longitudinally extending ridges separated by corresponding longitudinally extending valleys.

In a further embodiment, the continuous exterior surface of the body of the head, facial or ear cushion forms opposing first and second exterior sidewall surfaces of the body and the internal cavity of the head, facial or ear cushion is formed by an interior surface which extends around the internal cavity to form first and second interior sidewall surfaces, with the first and second exterior and interior sidewall surfaces defining corresponding first and second sidewalls of the body of the head, facial or ear cushion. One of the sidewalls of the body includes an access opening that extends longitudinally through the body from the exterior sidewall surface to the interior sidewall surface of the body to form overlapping sidewall flaps which define an access path through one of the first and second sidewalls of the body for inserting the strap of a respiratory or traditional device into the internal cavity of the head, facial or ear cushion and securing the head, facial or ear cushion to the strap.

The present invention further includes a head, facial or ear cushion for mounting to a strap of a respiratory or traditional device wherein the head, facial or ear cushion includes a rectangular body, and an adhesive strip located on an inner surface of the body and extending longitudinally parallel to and offset laterally from a longitudinal centerline of the body. The body of the head, facial or ear cushion is foldable into a folded configuration, along the longitudinal centerline of the body, to enclose the strap of the respiratory or traditional device and secure the strap in the folded configuration, by contact of the adhesive strip with opposing regions of the inner surface of the folded body.

In further embodiments, at least one corner of the head, facial or ear cushion may be rounded, and the body may further include an inwardly curved recess, centered approximately with respect to the separation line, to facilitate forming a curvature in body when the body is separated along the separation line, in order to provide a design which avoids the formation of strap/head, facial or ear cushion marks in the face of the patient, after wearing a medical respiratory or a traditional device or mask for a prolonged period of time.

### BRIEF DESCRIPTION OF THE DRAWINGS

The invention will now be described, by way of example, with reference to the accompanying drawings in which:

FIG. 1 is a diagrammatic side view illustration of a patient's head with a respiratory or traditional mask secured thereto by a strap;

FIG. 2A is a diagrammatic front elevational illustration of a head, facial or ear cushion in its flat, unfolded flat configuration;

FIG. 2B is a diagrammatic front elevational illustration of the head, facial or ear cushion in the folded, but not yet secured configuration;

FIG. 2C is an enlarged diagrammatic end view illustration of the head, facial or ear cushion in the folded, but not yet secured configuration of FIG. 2B;

FIG. 2D is an enlarged diagrammatic end view illustration of the head, facial or ear cushion shown in the final folded configuration and secured to a strap;

FIG. 2E is a diagrammatic front elevational illustration of the head, facial or ear cushion with an adhesive shown in the folded configuration and attached to a strap;

FIG. 2F is a diagrammatic illustration of the folded head, facial or ear cushion, separated along the separation or perforation line and curved so as to more closely conform with the upper curvature of the ear of the patient;

FIG. 3A is a diagrammatic side elevational view of a second embodiment of the head, facial or ear cushion according to the present invention;

FIG. 3B is a diagrammatic front elevational view of the second embodiment of the head, facial or ear cushion of FIG. 3A;

FIG. 4A is a diagrammatic side elevational view of a third embodiment of the head, facial or ear cushion according to the present invention;

FIG. 4B is a diagrammatic front elevational view of the third embodiment of the head, facial or ear cushion of FIG. 4A;

FIG. 5A is a diagrammatic side elevational view of a fourth embodiment of the head, facial or ear cushion according to the present invention;

FIG. 5B is a diagrammatic front elevational view of the fourth embodiment of the head, facial or ear cushion of FIG. 5A;

FIGS. 6A and 6B are respectively diagrammatic side elevational views of a fifth and sixth embodiment of the head, facial or ear cushion according to the present invention;

FIG. 7A is a diagrammatic side elevational view of a seventh embodiment of the head, facial or ear cushion according to the present invention;

FIG. 7B is a diagrammatic front elevational view of the seventh embodiment of the head, facial or ear cushion of FIG. 7A; and

FIG. 8 is a front elevational view showing modifications to the head, facial or ear cushion according to the present invention.

### DETAILED DESCRIPTION OF THE INVENTION

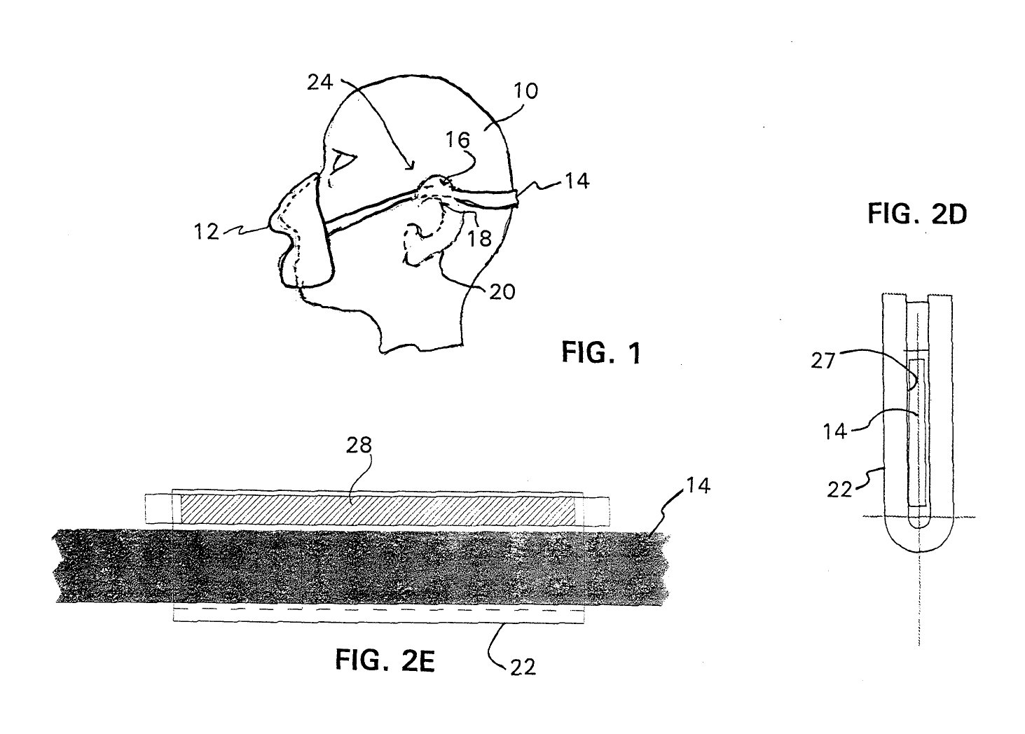

Referring to FIG. 1, therein is shown a diagrammatic illustration of a patient's head 10 with a respiratory or traditional mask 12 positioned over the patient mouth and nose region a strap 14 extending from one side of the mask 12 and around the back of the patient's head 10 to the other side of the mask 12. When passing around the back of the patient's head 10, the strap 14 passes over a vertically top most portion of the patient's outer ear, namely, an upper curvature 16 of the left outer ear 20, generally between the ear flap 18 and the side of the patient's head 10, and then extends around the back of the patient's head 10 and also passes over the upper curvature 16 of the patient's right outer ear 20, generally between the ear flap 18 and the side of the patient's head 10, which is not shown in the Figure.

As discussed herein above, in the prior art a region 24 of the strap 14 typically bears, presses, abrades and/or rubs against the upper curvature 16 of the ear 20, generally between the ear flap 18 and the side of the patient's head 10. Such pressure, scrapping, abrading and/or rubbing action generally results in, overtime, discomfort and/or irritation of the patient's skin, particularly in the area of upper curvature 16 of the patient's ear 20. This is compounded when such pressure, scrapping and/or rubbing is repeated multiple times each night, night after night. Such pressure, scrapping and/or rubbing can easily result in unbearable discomfort which may eventually lead to compliance issues or possibly complete discontinuance of the sleep apnea treatment procedure.

According to the present invention, the first and the second head, facial or ear cushions 22 are initially secured to the strap 14 and each of the first and the second head, facial or ear cushions 22 are adjusted along the length of the strap 14 so as to be positioned on a side of the patient's head 10 in the region 24 of the strap 14 which normally contacts or bears against the upper curvature 16 of the outer ear 20, generally between the ear flap 18 and the side of the patient's head 10. Thereafter, the opposed ends of the strap 14 are then attached to the mask 12 in a conventional manner. The first and the second head, facial or ear cushions 22 are thereby located so as to provide a cushioning engagement between the strap 14 and the patient's ears 20 and/or skin, in this area, and thereby minimize discomfort and/or irritation that the patient may experience when the mask 12 is worn for a prolonged period of time, e.g, each night for many, many years.

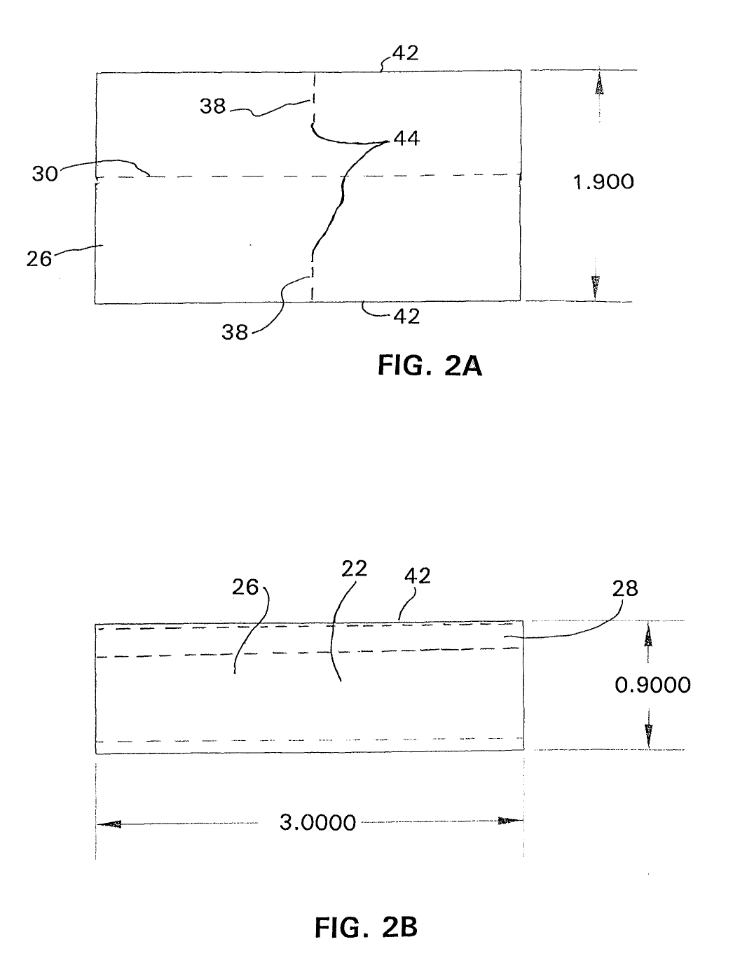

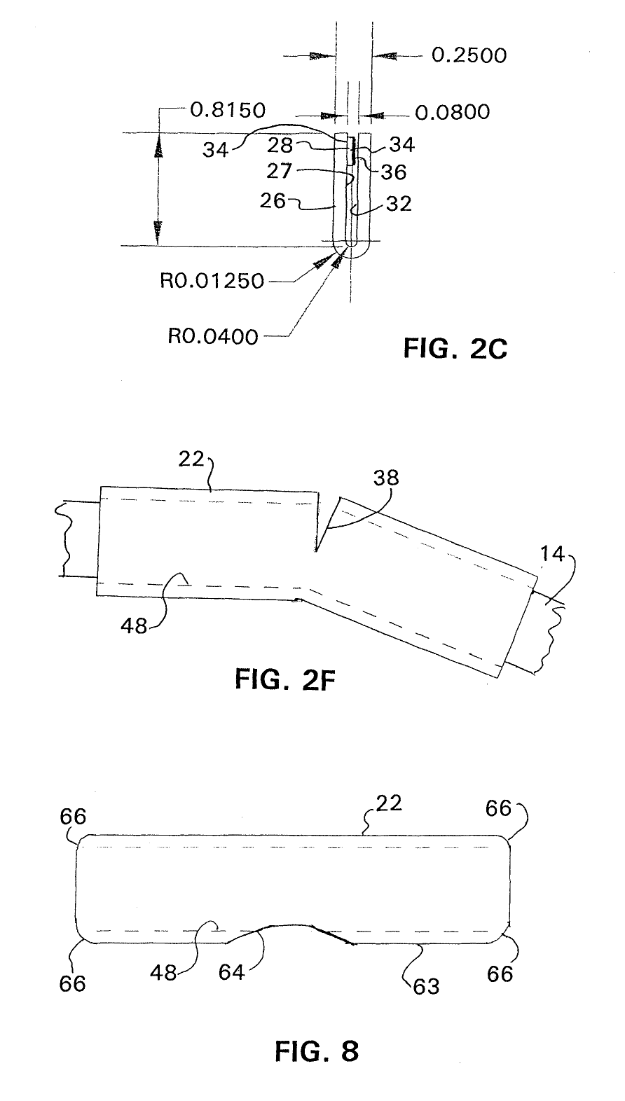

As illustrated in a first embodiment illustrated in FIGS. 2A, 2B, 2C, 2D and 2E, the head, facial or ear cushion 22, according to the present invention, generally comprises a body 26 having a generally rectangular profile or shape that is folded over a longitudinal line or axis to form an internal cavity 27 with an adhesive strip 28 located within the internal cavity 27 of the cushion 22 and along an inwardly facing surface 32 of the body 26. Generally, the adhesive strip 28 extends longitudinally parallel to and offset laterally from the longitudinal centerline 30 of the body 26. The adhesive strip 28 comprises, for example, an adhesive tape, having an adhesive 34 on both sides thereof, with one side of the adhesive tape being secured directly to the inwardly facing surface 32 of the body 26 of the head, facial or ear cushion 22, by the first adhesive 34 on that side of the tape, and the second adhesive 34 on the second opposite side of the tape, that is, on the exposed side of the adhesive tape, being covered by a peel away, removable layer 36. Alternately, the adhesive strip 28 may be an adhesive 34 which is applied directly to the inwardly facing surface 32 of the body 26, with the outer surface of the adhesive strip 28 again being covered with a peel away, removable layer 36.

According to a first embodiment, the body 26 of the head, facial or ear cushion 22 is approximately 3 inches long and 1.90 inches wide, in its flat, unfolded state as shown in FIG. 2A thereby having a width of approximately 0.90 inches and a thickness of about 0.25 of an inch when folded about a strap 14 so as to define the internal cavity 27 as described below and shown in FIGS. 2B, 2C, 2D and 2E, for example. The thickness of the head, facial or ear cushion 22 is designed so that the head, facial or ear cushion 22 can be lightly, but comfortably, sandwiched between the upper most portion of the ear lobe 20 of the patient and the side of the patient's head 10, without causing discomfort, and such sandwiching further assists with avoiding axial movement of the head, facial or ear cushion 22 along the strap 14. An exterior surface, located along the folded longitudinal edge of the head, facial or ear cushion 22, has a radius of curvature of about 0.125 of an inch.

Prior to use of the head, facial or ear cushion 22, and as illustrated in FIGS. 2C and 2D, the peel away removable layer 36 is removed from the adhesive strip 28 and then the body 26 is folded, along its longitudinal centerline 30, generally symmetrically around the strap 14 in the region 24 in which the strap 14 would normally abut or bear against the upper curvature 16 of the ear 20, generally between the ear flap 18 and the side of the patient's head 10, so that the adhesive strip 28 is brought into contact with the opposing region of inner surface 32 of the body 26, thereby forming the internal cavity 27 accommodating the strap 14 and securing the head, facial or ear cushion 22 to the strap 14 in the region 24 of the strap 14, as generally shown in FIG. 2E.

If desired, the body 26 of the head, facial or ear cushion 22 may be divided along a separation or perforation line 38 that extends from approximately a mid-point along an outer longitudinal edge 42 of the body 26, that is, the edge 42 of body 26 located away from the upper curvature 16 of the ear 20, and partially across the body 26 to an inner ending point 44 which is short of the longitudinal centerline 30 of the body 26. This separation or perforation line 38 promotes cutting, ripping or separation of the material of the body 26, along the separation or perforation line 38 prior to use. Alternatively, the head, facial or ear cushion 22 may be cut along the separation or perforation line 38 prior to packaging the head, facial or ear cushion 22.

According to one present embodiment of the head, facial or ear cushion 22, the separation or perforation lines 38 extends to an inner termination point 44 that may be short of the outer edge of the strap 14, that is, the edge of the strap 14 away from the upper curvature 16 of the outer ear 20, but the separation or perforation lines 38 may extend further, if so desired. As indicated, the cutting, ripping or otherwise separating the body 26, along the separation or perforation line 38, allows the body 26 of the head, facial or ear cushion 22 to assume a slightly curved shape or configuration which more closely conforms with to the upper curvature 16 of the outer ear 20, between the ear flap 18 and the side of the patient's head 10, as shown in FIG. 2F. Such curvature of the head, facial or ear cushion 22 thereby further ensures that the head, facial or ear cushion 22 will remain in its installed and desired position along the strap 14, i.e., the head, facial or ear cushion 22 will avoid sliding or axial movement along the strap 14 during use, thereby reducing the possibility that the head, facial or ear cushion 22 will not provide its intended cushioning effect for the upper curvature 16 of the outer ear 20, between the ear flap 18 and the side of the patient's head 10 during use, and minimize discomfort and irritation of the patient.

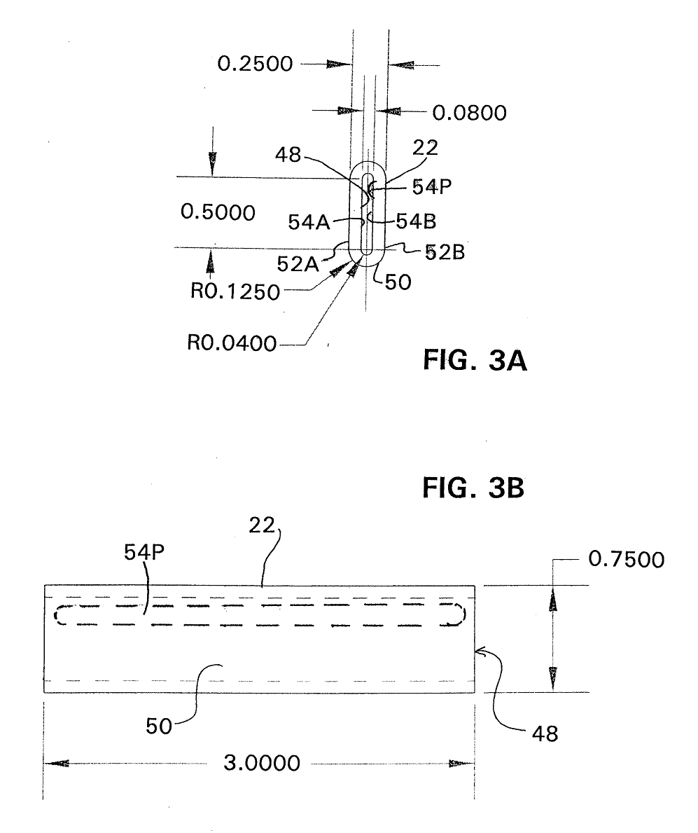

With reference now to FIGS. 3A and 3B, a description concerning an alternative embodiment of the head, facial or ear cushion 22 will now be discussed. According to this first embodiment, and as shown in FIG. 3A, the body 26 of the head, facial or ear cushion 22 comprises a unitary body having a generally oval cross section that encloses and defines an internal cavity 48 which extends longitudinally completely through body 26 and this internal cavity 48 is sized to closely receive and accommodate the strap 14 of the mask 12, during use, with a small clearance. As shown, an external surface 52 of the body 26 forms opposed first and second exterior sidewall surfaces 52A and 52B and internal cavity 48 is defined by an interior surface 54 which extends around internal cavity 48 and forms interior sidewall surfaces 54A and 54B. The cavity 48 is sized so as to closely receive and accommodate the strap 14, and minimize axial movement of the head, facial or ear cushion 22 along the strap 14 during use of the head, facial or ear cushion 22. That is, the opposed inwardly facing surfaces 54A and 54B of the internal cavity 48 engage with the strap 14 and generally restrict axial movement of the elongate body along the strap 14. If desired, one or more bumps, protrusions, nubs, nodes, a piece of adhesive or some other frictional inducing feature, generally depicted as element 54P, may be applied to or otherwise carried by or formed in one or more of the inwardly facing surfaces 54A and 54B of the internal cavity 48 and configured so as to protrude into a surface of the strap 14 and further resist sliding movement of the head, facial or ear cushion 22 with respect to the strap 14. It is to be appreciated that the frictional inducing feature 54P can have a variety of shapes, sizes and/or length which helps resist sliding movement of the head, facial or ear cushion 22 with respect to the strap 14.

As diagrammatically shown in FIG. 3B, the body 26 of the head, facial or ear cushion 22 is approximately 3 inches long, about 0.75 of an inch wide and about 0.25 of an inch thick. As noted above, the thickness of the head, facial or ear cushion 22 is designed so that the head, facial or ear cushion 22 can be lightly, but comfortably, sandwiched between the upper ear lobe of the patient and the side of the patient's head 10, without causing discomfort, and such sandwiching further assists with avoiding axial movement of the head, facial or ear cushion 22 along the strap 14. The thickness of the internal cavity 48 is approximately 0.08 of an inch and a width of the internal cavity 48 is approximately 0.58 of an inch. The exterior surface 52 at each opposed longitudinal edge of the head, facial or ear cushion 22 has a radius of curvature of about 0.125 of an inch. The head, facial or ear cushion 22 typically has a thickness of between about 3/32 of an inch to about ½ of an inch, typically about ¼ of an inch.

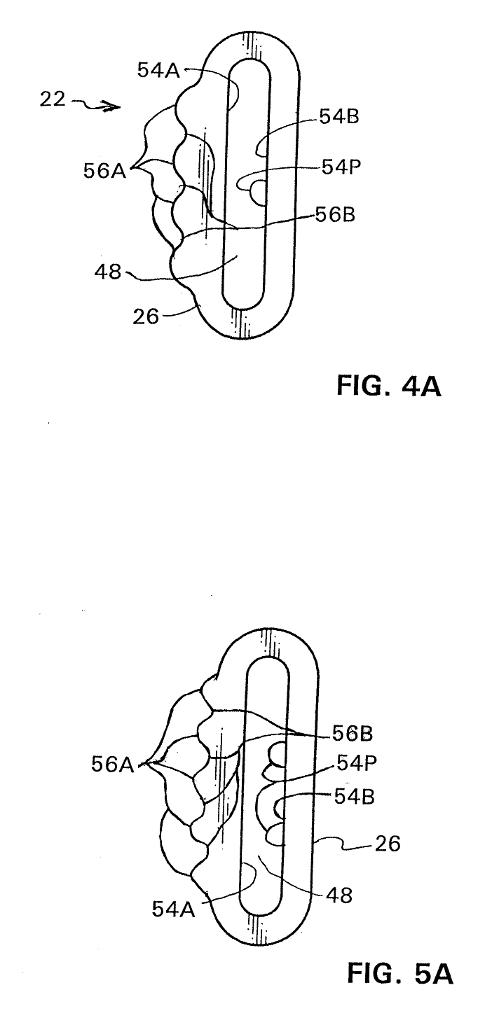

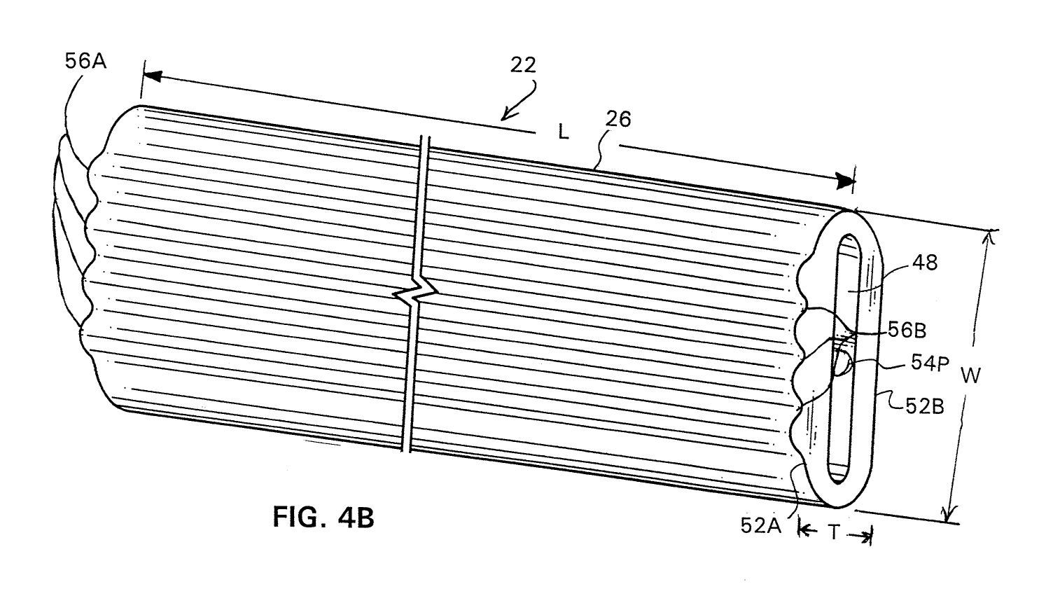

Further embodiments of the invention will now be described with reference to FIGS. 4A and 4B, 5A and 5B and 6A and 6B. As these embodiments are all quite similar to the above discussed embodiment illustrated in FIGS. 3A and 3B, for example, only the differences between these embodiments and the previous embodiment of FIGS. 3A and 3B will be discussed in detail.

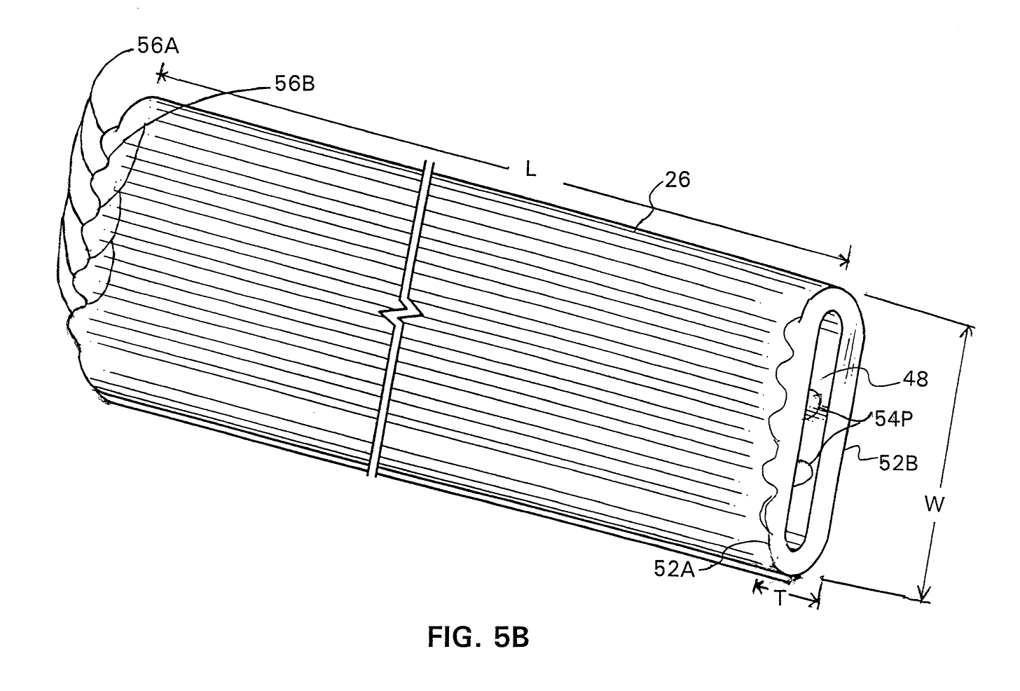

With respect to the embodiments of the head, facial or ear cushion 22 illustrated in FIGS. 4A-6B in general, in order to provide the desired cushioning effect to the patent and to space a main portion of the body 26 of the head, facial or ear cushion away from the skin of the patient, a plurality of longitudinally extended ribs are provided in at least one of the exterior sidewalls 52A and/or 52B of the head, facial or ear cushion 22. As can be seen in FIGS. 4A and 4B and FIGS. 5A and 5B, at least one of exterior sidewall surfaces 52A and 52B has a plurality of longitudinally extending elongate nubs or ridges 56A which are each separated from one another by a parallel longitudinally extending valley 56B. The ridges 56A are designed to minimize the surface contact, between the head, facial or ear cushion 22 and the patient's skin, while promoting circulation in the head/facial area of the patient wearing the head, facial or ear cushion 22. That is, the ridges 56A assist with adequately spacing the main portion of the body 26 of the head, facial or ear cushion 22 a small distance away from the skin of the patient, during use, which improves the overall comfort of the head, facial or ear cushion 22 to a patient wearing the same. According to the embodiment of FIGS. 4A and 4B, at least one of the exterior sidewall surfaces 52A or 52B of the head, facial or ear cushion 22 is provided with four ridges 56A, while according to the embodiment of FIGS. 5A and 5B, at least one of the exterior sidewall surfaces 52A or 52B of the head, facial or ear cushion 22 is provided with five longitudinally extending ridges 56A. It is to be appreciated that the number of ridges 56A provided on each or both of the exterior sidewall surfaces 52A or 52B of the head, facial or ear cushion 22 as well as the spacing between each adjacent pair of ridges 56A can vary depending upon the particular application. In addition, the height or each of the ridges 56A as well as the depth of each of the longitudinally extending valley 56B can vary depending upon the particular application.

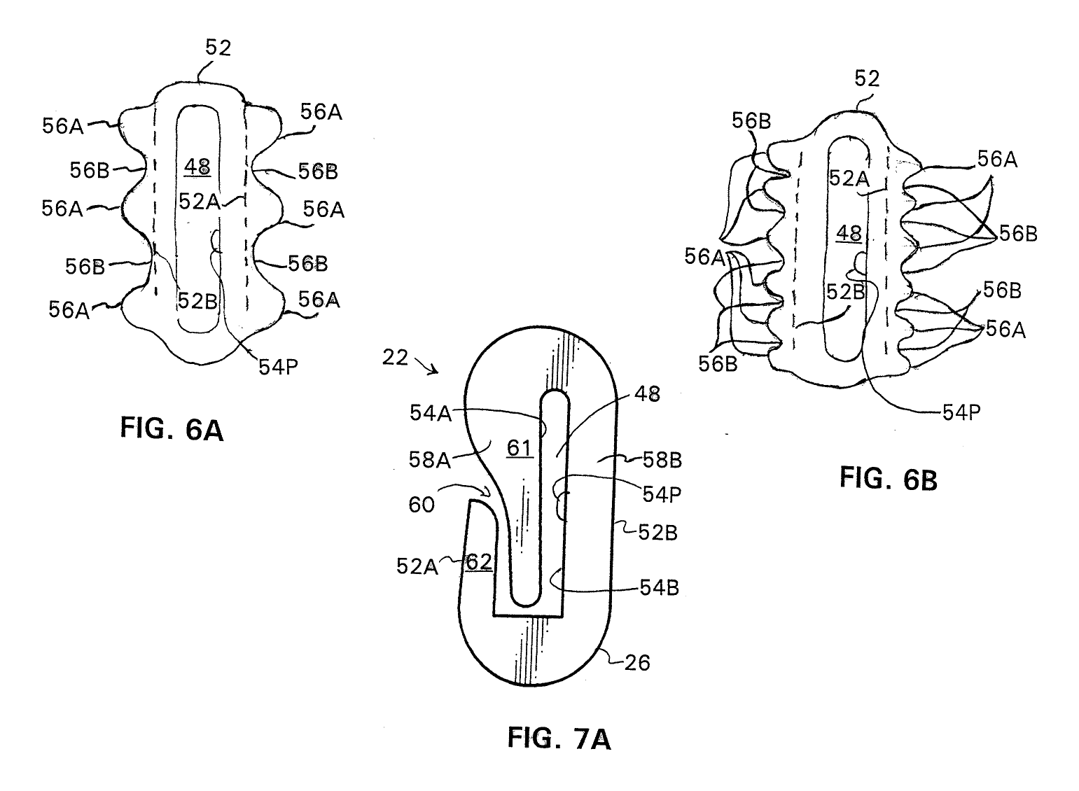

FIGS. 6A and 6B, in turn, illustrate an embodiment of the head, facial or ear cushion 22 in which both opposed exterior sidewall surfaces 52A and 52B of the head, facial or ear cushion 22 are each provided with corresponding longitudinal ridges 56A and valleys 56B—FIG. 6A illustrates an embodiment having three longitudinal ridges 56A—with the valleys 56B located therebetween—on each of the opposed exterior sidewall surfaces 52A and 52B of the head, facial or ear cushion. FIG. 6B illustrates an embodiment having six longitudinal ridges 56A—with the valleys 56B located therebetween—on each of the opposed exterior sidewall surfaces 52A and 52B.

It is to be appreciated that the number of ridges 56A provided on each or both of the exterior sidewall surfaces 52A or 52B of the head, facial or ear cushion 22 as well as the spacing between each adjacent pair of ridges 56A can vary depending upon the particular application. In addition, the height of each of the ridges 56A as well as the depth of each of the longitudinally extending valley 56B can also vary depending upon the particular application. It is to be appreciated that the embodiments of FIGS. 6A and 6B eliminate the need to orient the head, facial or ear cushion 22 in any particular orientation with respect to the strap 14 when affixing the pair of head, facial or ear cushions 22 to the strap 14, e.g., one set of ridges 56A and valleys 56B is always properly oriented and positioned so as to engage with the head, facial or ears of the patient during use. As noted above, the ridges 56A and valleys 56B create a space between the main portion of the body 26 of the head, facial or ear cushion 22 and the patient's head 10 which generally promote's the circulation of air between the head, facial or ear cushion 22 and the skin of both the patient's head and the patient's ear, thereby increasing circulation in that region and improving the patient's comfort as well as the wear characteristics of the head, facial or ear cushion 22 while avoiding the formation of “wear marks” in the skin of the patient from prolonged use of the same. In addition, it will also be appreciated that the presence of the ridges 54A and the valleys 54B, on both opposes exterior sidewall surfaces 52A and 52B of the head, facial or ear cushion 22, allows the head, facial or ear cushion 22 to be rotated 180 degrees about and relative to the strap 14, when one set of the ridges 54A becomes sufficiently worn, thereby prolonging the use of the head, facial or ear cushion 22 before having to replace the same. The location and spacing of the ridges 56A with respect to the valleys 56B are designed to avoid the formation of strap/head, facial or ear cushion marks in the face of the patient after wearing a medical respiratory or traditional device for a prolonged period of time, e.g., overnight.

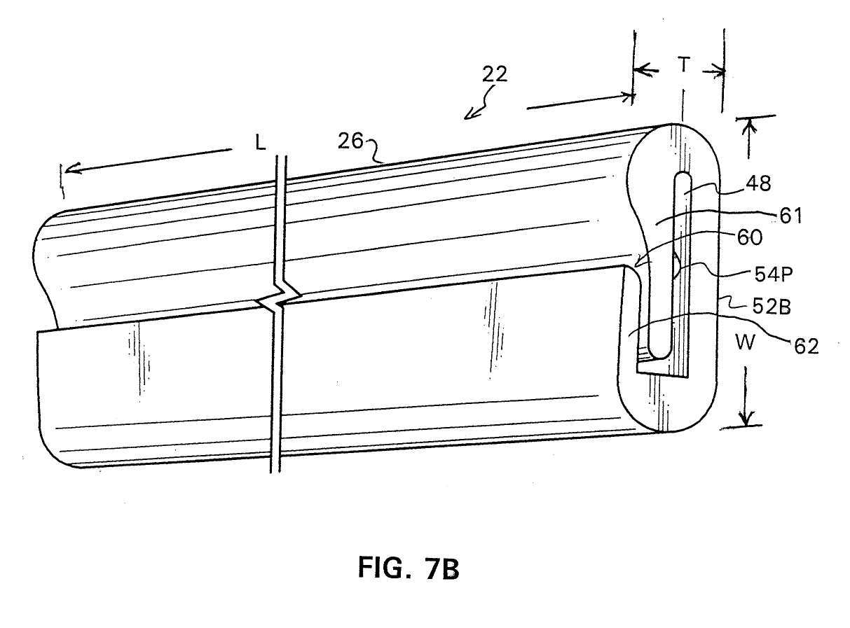

With reference now to FIGS. 7A and 7B, a seventh embodiment of the present invention will now be discussed. According to this embodiment, it is not necessary to attach the head, facial or ear cushions to the strap 14 prior to the strap 14 being connected to the mask 12. That is, according to the second through the sixth embodiments discussed above, at least one end of the strap 14 must be unconnected to the mask 12 so that at least that free unconnected end of the strap 14 can pass through the internal cavity 27, 48 of the first and the second head, facial or ear cushions 22 in order to secure the head, facial or ear cushions 22 to the strap 14. Thereafter, the unconnected end of the strap 14 can be then connected/reconnected with the mask 12, in a conventional manner, and each of the first and the second head, facial or ear cushions 22 slid along the strap 14 into their desired axial locations along the strap 14 so each of the first and the second head, facial or ear cushions 22 are correctly positioned to engage with the upper curvature 16 of the outer ear 20, between the ear flap 18 and the side of the patient's head 10, during use, and provide the desired cushioning effect. As noted above, the embodiment of FIGS. 7A and 7B allows the head, facial or ear cushions 22 to be affixed to a strap 14 without requiring at least one end of the strap 14 to be unconnected with the mask 12, thereby facilitating both the subsequent affixing of the head, facial or ear cushions 22 to the strap 14 as well as replacement of a worn or damaged head, facial or ear cushion 22 without requiring disconnection of the strap 14 from the mask 12.

According to the embodiment of FIGS. 7A and 7B, the body 26 of the head, facial or ear cushion 22 comprises a generally oval transverse cross sectional shape that encloses and defines an internal cavity 48 which extends longitudinally completely through the body 26 from one end to the other opposite end, as with the previous embodiments. The internal cavity 48 is also sized to closely receive and accommodate the strap 14 of the mask 12 therein during use with a small amount of clearance, as with the previous embodiments. As shown, the head, facial or ear cushion 22 has an exterior surface 52 which extends around and thus generally surrounds and/or encloses the body 26 and forms opposed exterior sidewall surfaces 52A and 52B. The internal cavity 48 is defined by an interior surface 54 which extends generally around internal cavity 48 to form interior opposed sidewall surfaces 54A and 54B. The internal cavity 48 has a small clearance with the strap 14 so as to minimize axial movement of the head, facial or ear cushion 22 along the strap 14 during use of the head, facial or ear cushion 22. That is, the opposed inwardly facing surfaces 54A and 54B of the internal cavity 48 typically engage with the strap 14 and generally restrict axial movement of the elongate body along the strap 14. If desired, one or more bumps, protrusions, nubs, nodes, a piece of adhesive or some other frictional inducing element, generally depicted as element 54P, may be applied to or otherwise carried by or formed in one or more of the inwardly facing surfaces 54A and 54B of the internal cavity 48 and configured so as to protrude into a surface of the strap 14 and further resist sliding movement of the head, facial or ear cushion 22 with respect to the strap 14. It is to be appreciated that the frictional inducing element 54P can have a variety of shapes, sizes and/or length which helps resist sliding movement of the head, facial or ear cushion 22 with respect to the strap 14.

As shown in FIG. 7B, a first one of the sidewalls 58A of the body 26 is generally thicker than a second opposed sidewall 58B of the body 26. That is, the spacing between the first external sidewall surface 52A and the corresponding first internal sidewall surface 54A of the first sidewall 58A is greater than the spacing between the second external sidewall surface 52B and the corresponding second internal sidewall surface 54B of the second sidewall 58B. The additional thickness of the first sidewall 58A permits an access opening 60 to be easily and conveniently formed in the first sidewall 58A during subsequent manufacture of the head, facial or ear cushion 22. Typically the access opening 60 is cut or otherwise formed by a “water jet” process in which the jetted water removes a portion of the first sidewall 58A of the body 26 so as to form the access opening 60 therein. It is to be appreciated that the access opening 60 must extend along the entire longitudinal length of the body 26 of the head, facial or ear cushion 22, from the first end to the second opposed end thereof and also from the first external sidewall surface 52A completely through to the corresponding first internal sidewall surface 54A of the first sidewall 58A. As shown in FIGS. 7A and 7B, the access opening 60 is generally formed or cut into the first sidewall 58A at a shallow angle, e.g., about 5 degrees to about 20 degrees, with respect to the external sidewall surface 52A and extends toward a base of the body 26. Thereafter, the access opening 60 extends laterally across the base, generally normal to the first internal sidewall surface 54A of the first sidewall 58A, until reaching a base of the internal cavity 48. That is, the access opening 60 is generally L-shaped prior to merging with the internal cavity 48.

As shown in FIG. 7A, the access opening 60 and the internal cavity 48 together form a generally U-shaped passageway which is configured and sized so as to allow the strap 14 of the mask 12 to pass through the access opening 60 and be received by and within the interior cavity 48 of the body 26, thereby eliminating the need to detach the strap 14 from the mask 12 in order to install the head, facial or ear cushion 22 on the strap 14 or replace a worn head, facial or ear cushion. Due to the formation of the access opening 60 in the first sidewall 58A of the body 26, the first sidewall 58A is divided into inner and outer sidewall sections 61, 62 which overlap one another and assist with retaining the strap 14, once accommodated within the internal cavity 48, and prevent inadvertent removal of the head, facial or ear cushion 22 from the strap 14. As shown in FIGS. 7A and 7B, the inner sidewall section 61 extends generally the entire height of the cavity 48 and is overlapped by the outer sidewall section 62 which typically extends over a distance of about one half of the entire height of the cavity 48 such that the access opening 60 is formed in a central region of the sidewall. Generally the shape of one or both of the inner and outer sidewall sections 61, 62 is altered somewhat during insertion of a strap 14 into the cavity 48. In addition, and as a result of such arrangement, the head, facial or ear cushion 22 can be releasably secured to the strap 14, typically without any adhesive, or other fastener, and can also be easily removed from the mask 12 and the strap 14 when desired or when replacement of the head, facial or ear cushion 22 is warranted.

According to this embodiment, the head, facial or ear cushion 22 has a width W of about 27/32 to 30/32 inch or so, a length L of about 3 inches and a thickness T of about 10/32 of an inch or so. It will also be appreciated that at least the sidewall 58A or 58B which does not include the access opening 60 may include corresponding ridges 56A and valleys 56B as described above, for the purposes and benefits described herein above.

According to still further modifications of the head, facial or ear cushion 22 as illustrated in FIG. 8, a bottom longitudinal edge 63 of the body 26 may include an inwardly curved recess 64, which is centered with respect to the length L of the head, facial or ear cushion 22 and this inwardly curved recess 64 promotes retention of the head, facial or ear cushion 22 at a desired axial location along the strap 14. The inwardly curved recess 64 forms a small curvature for engagement with the top most portion of the ear 20 of the patient and such engagement facilitates maintaining the head, facial or ear cushion 22 substantially centered with respect to the upper curvature 16 of the outer ear 20, between the ear flap 18 and the side of the patient's head 10, during use. It is to be appreciated that all of the exposed edges, of the inwardly curved recess 64, are generally sufficiently rounded or otherwise contoured so as to avoid forming any cutting or scrapping edge(s) or surface(s) which may create discomfort to the patient during use. If the inwardly curved recess 64 is also used in combination with the separation or perforation lines 38, the inwardly curved recess 64 also facilitates bending or curving of the body 26 when the body 26 is cut, ripped or otherwise separated, along the separation or perforation lines 38, so that the head, facial or ear cushion 22 more closely conforms with the upper curvature 16 of the outer ear 20, between the ear flap 18 and the side of the patient's head 10.

According to a further implementation of the head, facial or ear cushion 22, and as also illustrated in FIG. 8, at least one corner 66 of the body 26 of the head, facial or ear cushion 22 may be curved or otherwise rounded to further decrease the possibility of such corner(s) potentially causing patient discomfort and/or irritation, during use of the head, facial or ear cushion 22. Preferably at least two or all four of the corners 66 of the head, facial or ear cushion 22 are curved, rounded or otherwise contoured so as to decrease the likelihood of an exposed edge of the head, facial or ear cushion 22 possibly poking or jabbing into the skin of the patient during use and causing discomfort.

It will be noted that each head, facial or ear cushion 22 is designed to be worn for a number of nights before any head, facial or ear cushion 22 becomes sufficiently worn, frayed, torn or otherwise requires replacement. That is, each head, facial or ear cushion 22 generally will be worn for a plurality of days, or possibly a few weeks or so before requiring replacement. Moreover, it is to be appreciated that each head, facial or ear cushion 22 has a sufficient axial length L so that the inwardly facing surfaces 54A, 54B of the head, facial or ear cushion 22 sufficiently frictionally engage with the exterior surfaces of the strap 14 so as to inhibit generally axial movement of the head, facial or ear cushion 22 along the strap 14.

Also according to the present invention, the head, facial or ear cushion 22 is generally thinner than its length L and its width W, e.g., the head, facial or ear cushion 22 generally has a “flattened” oval shape which results in two elongate opposed exterior sidewall surfaces 52A and 52B of the head, facial or ear cushion 22 being generally planar and extending substantially parallel to one another and located fairly close to one another. That is, the two elongate opposed exterior surfaces 52A and 52B of the head, facial or ear cushion 22 are generally planar and extend parallel to one another so that the width W of the head, facial or ear cushion 22 is at least twice, and preferably about three to four times greater than, the thickness T of the head, facial or ear cushion 22. Due to such flattened arrangement, the two elongate opposed exterior surfaces 52A and 52B of the head, facial or ear cushion 22 can be conveniently located, during use, between the upper portion of the ear lobe 20 of the patient and the adjacent side of the patient's head 10 without causing discomfort to the patient. As a result of such arrangement, the two elongate opposed exterior surfaces 52A and 52B of the head, facial or ear cushion 22 are located so that one exterior surface 52A or 52B of the head, facial or ear cushion 22 abuts against the adjacent surface of the ear lobe 20 of the patient while the other exterior surface 52B or 52A of the head, facial or ear cushion 22 abuts against the adjacent skin on the side of the patient's head 10. The head, facial or ear cushion 22 has a thickness T which is designed so that the head, facial or ear cushion 22 can be lightly, but comfortably, sandwiched between the upper ear lobe of the patient and the side of the patient's head 10 so as to assist with avoiding axial movement of the head, facial or ear cushion 22 along the strap 14.

According to the present invention, the head, facial or ear cushion 22 is, for example, typically manufactured from either a soft foam material, a soft cloth, a closed cell chemically crossed linked polyethylene bun foam or other similar material, which is suitable for providing a cushioned, flexible, relatively smooth and relatively non-abrasive surface which bears against the ear 20 and/or skin of the patient so as to avoid discomfort and irritation to the patient. A few possibly closed cell chemically crossed linked polyethylene bun foams are supplied by Sekisui Voltek of Lawrence, Mass. as product identifications numbers Minicel L200, L300 and L380 which have densities of 2.0 pcf, 3.0 pcf and 3.8, respectively, and Minicel L200 is the preferred foam due to its lower density. In the embodiments illustrated in FIGS. 2A-E and 3A and 3B, for example, the adhesive strip 28 typically extends the full length of the body 26 and has a width of typically between about 0.15 of an inch and 0.35 of an inch and is typically about 0.25 of an inch.

It is to be appreciated, that although the above description discusses the use of a pair of head, facial or ear cushions 22, the use of more of less head, facial or ear cushions 22 is possible. For example, the use of a third and possibly a fourth head, facial or ear cushion(s), located between the pair of head, facial or ear cushions 22, may be secured to the strap 14 and located for engaging with the back of the patient's head 10 so as to avoid irritation to the scalp and/or possibly also avoid unwanted tugging or pulling of the patient's hair by the strap 14 during use of the mask 12. Alternatively, a single longer elongate head, facial or ear cushion may be utilized which is substantially long enough to extend generally along the strap 14 from adjacent a first connection of the strap 14 with the mask 12 to adjacent a second opposite connection of the strap 14 with the mask 12. This single elongate head, facial or ear cushion generally avoids or minimizes irritation to the patient's cheeks, ears 20 as well as the hair and the back of the patient's head 10. It is to be appreciated when a pair of head, facial or ear cushions 22 are utilized, if desired, each one of the pair of head, facial or ear cushions 22 can extend a longer distance along the strap 14, either in the direction toward of the mask 12 or away from the mask 12, so as to cover a greater surface area of the strap 14 and thereby generally avoid or minimize irritation to the patient's cheeks, ears 20, hair and/or back of the patient's head 10.

Since certain changes may be made in the above described head, facial or ear cushion for a strap of a respiratory or traditional device, without departing from the spirit and scope of the invention herein involved, it is intended that all of the subject matter of the above description or shown in the accompanying drawings shall be interpreted merely as examples illustrating the inventive concept herein and shall not be construed as limiting the invention.

Wherefore, I/We claim:

1. A cushion for mounting to a strap of a respiratory device, the cushion comprising:
an elongate flattened body defining an internal cavity for accommodating the strap of the respiratory device,
the cushion generally having a flattened oval shape forming two elongate opposed exterior surfaces being generally planar and extending substantially parallel to one another so that a width of the cushion is at least twice a thickness of the cushion, and
the elongate body being configured so as to restrict axial movement of the body along the strap, during use, and maintain the cushion substantially centered with respect to an upper curvature of an ear, between an ear flap of the ear and a side of a head of a patient.

2. The cushion according to claim 1, wherein the body is folded into a folded configuration, about a longitudinal centerline of the body, to define the internal cavity and at least partially enclose the strap of the respiratory device, and the cushion is retained in its folded configuration by an adhesive engaging with opposed inwardly facing surfaces of the folded body.

3. The cushion according to claim 2, wherein the adhesive is an adhesive strip, which extends longitudinally along one of the inwardly facing surfaces of the internal cavity parallel to, but laterally offset and spaced from, the longitudinal centerline of the body, to facilitate engagement with an opposed inwardly facing surface of the folded body and retain the folded body in the folded configuration.

4. The cushion according to claim 2, wherein an outwardly facing surface of the adhesive has a removable layer which covers the adhesive, and the removable layer is removed prior to installing the cushion on desired strap so that the adhesive adhesively engages with an opposed inwardly facing surface of the folded body and retains the folded body in the folded configuration.

5. The cushion according to claim 1, wherein the cushion has perforation lines which extend from mid-point along each outer longitudinal edge of the body toward the longitudinal centerline, and the perforation lines terminating short of the longitudinal centerline of the body.

6. The cushion according to claim 5, wherein, prior to installing the cushion on desired strap, the cushion is separated along the perforation lines to form a curvature in the cushion which generally conforms to the upper curvature of the ear, between the ear flap of the ear and the side of the head of the patient.

7. The cushion according to claim 1, wherein at least one corner of the cushion is rounded to avoid irritation to skin of the patient during use.

8. The cushion according to claim 1, wherein the cushion is secured to the strap and centered with respect to a region of the strap which bears against the upper curvature of the ear, between the ear flap of the ear and the side of the head of the patient.

9. The cushion according to claim 1, wherein a width of the internal cavity is slightly wider than a width of the strap to be accommodated so that inwardly facing surfaces of the internal cavity engage with the strap and restrict axial movement of the body along the strap.

10. The cushion according to claim 1, wherein the body further includes an inwardly curved recess, generally centered with respect to a longitudinal length of the body, and the inwardly curved recess assists with maintaining the cushion substantially centered with respect to the upper curvature of the ear, between the ear flap of the ear and the side of the head of the patient.

11. The cushion according to claim 1, wherein the flattened body is generally a flattened oval shape which has a continuous exterior surface which extends continuously and completely around the body so as to encase the strap, and the internal cavity extends completely through the body.

12. The cushion according to claim 11, wherein the cushion has perforation lines which extend from mid-point along each outer longitudinal edge of the body toward the longitudinal centerline, and the perforation lines terminating short of the longitudinal centerline of the body; and
prior to installing the cushion on desired strap, the cushion is separated along the perforation lines to form a curvature in the cushion which generally conforms to the upper curvature of the ear, between the ear flap of the ear and the side of the head of the patient.

13. The cushion according to claim 11, wherein the cushion is secured to the strap and centered with respect to a region of the strap which bears against the upper curvature of the ear, between the ear flap of the ear and the side of the head of the patient.

14. The cushion according to claim 12, wherein the body further includes an inwardly curved recess, centered approximately with respect to the perforation line which further assists with forming the body into the curved configuration when the body is separated along the perforation lines.

15. The cushion according to claim 11, wherein the body further includes an inwardly curved recess which is generally centered with respect to a longitudinal length of the body, and the inwardly curved recess engages with the ear and assists with maintaining the cushion substantially centered with respect to the upper curvature of the ear, between the ear flap of the ear and the side of the head of the patient.

16. The cushion according to claim 1, wherein the two elongate opposed exterior surfaces of the cushion are generally planar and extend substantially parallel to one another so that the width of the cushion is between about three and four times the thickness of the cushion.

17. The cushion according to claim 11, wherein the continuous exterior surface forms opposing facing first and second exterior sidewall faces and at least one of the exterior sidewall surfaces includes a plurality of longitudinally extending ridges which are separated from one another by a corresponding longitudinally extending valley.

18. The cushion according to claim 11, wherein the continuous exterior surface forms opposing facing first and second exterior sidewall faces and both of the exterior sidewall surfaces include a plurality of longitudinally extending ridges which are separated from one another by a corresponding longitudinally extending valley.

19. The cushion according to claim 11, wherein the continuous exterior surface forms opposing facing first and second exterior sidewall surfaces of the body and the internal cavity is formed by an interior surface which extends around internal cavity to form first and second interior sidewall surfaces with the first and second exterior and interior sidewall surfaces defining corresponding first and second sidewalls of the body of the cushion, and
one of the first and second sidewalls of the body includes an access opening which extends longitudinally the length of the body and between the corresponding exterior and interior sidewall surfaces of the body to form overlapping sidewall flaps defining the access opening through the one of the first and second sidewalls of the body for passing the strap of a respiratory device into the internal cavity of the cushion to secure the cushion to the strap.

20. A cushion for mounting to a strap of a respiratory device, the cushion comprising:
a rectangular body; and
an adhesive strip located on an inner surface of the body and extending longitudinally parallel to and offset laterally from a longitudinal centerline of the body, wherein
the body is foldable into a folded configuration along the longitudinal centerline of the body to enclose the strap of the respiratory device and secured in the folded configuration by contact of the adhesive strip to between opposing regions of the inner surface of the folded body.
